(19) United States
(12) Patent Application Publication
(10) Pub. No.: US 2026/0354760 A1
(43) Pub. Date: Nov. 12, 2026

(54) AN APPARATUS AND METHOD FOR USE IN GUIDING OR FACILITATING THE INSERTION OF AN ELONGATED MEDICAL DEVICE INTO ANATOMICAL OPENINGS

(71) Applicant: GRAVIDA HEALTH PTY. LTD., WHEELERS HILL, VICTORIA (AU)

(72) Inventors: Densearn SEO, DONCASTER (AU); Ritesh Rikain Satyajit WARTY, BURWOOD EAST (AU); Vinayak SMITH, WHEELERS HILL (AU)

(73) Assignee: GRAVIDA HEALTH PTY. LTD., WHEELERS HILL, VICTORIA (AU)

(21) Appl. No.: 19/124,982

(22) PCT Filed: Oct. 27, 2023
(86) PCT No.: PCT/AU2023/051092

§ 371 Date: Apr. 28, 2025

(30) Foreign Application Priority Data
Oct. 28, 2022 (AU) 2022903201

(51) Int. Cl. A61B 5/00; A61B 17/42; A61M 25/01

(52) U.S. Cl. CPC A61B 5/6826; A61M 25/01; A61B 17/42; A61M 2205/586; A61M 2250/00

(57) ABSTRACT

An apparatus for guiding or facilitating the insertion of an elongated medical device into anatomical openings of humans and animals. A catheter can form the medical device held by a hand controlled body comprising of a hand connector for connection to the operator's hand and a device connector that is to substantially capture the catheter and moved with the hand connector. When in position an extension and detent mechanism, which could in one form be a slide mechanism, extends the catheter to protrude a predetermined distance.

### FIELD OF THE INVENTION

The present invention relates to an apparatus and method for guiding or facilitating the insertion of medical devices into anatomical openings.

“Medical devices” throughout this document refers to catheters, balloons, and other tubes, wires, rods, threads, or similar elongated medical devices that are used for insertion into anatomical openings of humans and animals, or parts thereof, in vivo, ex vivo, post-mortem or in vitro, whether the anatomical openings are naturally, surgically or otherwise formed.

The invention has been developed primarily for use in obstetrics and gynaecology and will be described hereinafter with reference to these applications. However, it will be appreciated that the invention is not limited to this particular field of use but can be used in other medical, veterinary, forensic, scientific and healthcare uses.

### BACKGROUND OF THE INVENTION

There are a number of clinical procedures requiring the insertion of elongated medical devices, which are tube-like or wire-like objects, into anatomical openings. This can cause discomfort for the patient. Examples of such procedures include:
a) urinary catheterisation through the urethra to drain the bladder of urine in patients having limited or impaired mobility or who are experiencing urinary retention;
b) embryo transfer during in-vitro fertilisation (IVF);
c) Bakri@ balloon/balloon tamponade insertion for management of postpartum haemorrhage (PPH);
d) tubal flushing/patency testing during hystero-contrast-sonography (HyCoSy) or hysterosalpingography (HSG) or
e) fetal scalp electrode (FSE) insertion;
f) intrauterine pressure catheter (IUPC) insertion during labour to monitor uterine contractions;
g) Intrauterine insemination (IUI) for artificial insemination; or
h) mechanical induction of labour (IOL), in which labour is induced using a balloon catheter that can be inserted through the vagina into the cervix and/or the uterus.

For the mechanical IOL procedure especially, there are two common techniques for inserting the balloon into the cervix: via finger-based (digital) or speculum-based insertion.

With the former technique, the clinician performs a vaginal examination to locate the external os of the cervix. Following that, the clinician will then guide the balloon into the cervix along the gloved finger. This is an approximate procedure into target anatomical region and requires hand manipulation while also having to control and feed the catheter into location.

In the second technique, the speculum is used to maintain dilation of the vaginal canal so that the clinician can guide the balloon into the target anatomical region using a forceps whilst maintaining direct visualisation of the cervix. However, the use of a speculum insertion has been associated with increased procedural time compared to digital insertion, as well as being less tolerable. Though both have near identical success rates, digital insertion also requires less equipment and consumables, which could make it the preferred technique for those who are adept at the procedure. However to date there needs to be an improvement.

Under the existing clinical workflow for digital insertions as shown in FIG. 1, the patient is brought into a birthing suite, which acts as the site for the procedure. A cervical assessment is performed to assess the Bishop's score, which is a calculation used to predict whether induction of labor will be required to stimulate vaginal birth.

The clinician then prepares the cervix by cleaning it with an antiseptic solution and preparing the balloon catheter and water-filled syringe. The clinician performs a vaginal examination to locate the external os of the cervix. Following that, the clinician will then guide the balloon into the cervix along the gloved finger. The mechanical IOL procedure ends when the clinician confirms placement of the inflated balloon catheter within the target anatomical region and taping of the catheter end externally on the patient.

To date, there is only one similar device that has been identified in patent application no. US20210275100A1. This discloses a two-clip manipulator which attaches to and allows a sheath to be positioned inside the cervix and a catheter to thereby be inserted through the sheath and be positioned in a desired location in the uterus. In various embodiments of the invention, the manipulator may be attached or permanently connected to the sheath. In various embodiments of the invention, the sheath is fenestrated to allow the catheter to be detached from the sheath. In various embodiments of the invention, the manipulator allows the sheath to be positioned through the cervix canal to allow for catheter transmitted intrauterine pressure monitoring or balloon catheter assisted ripening of the cervix.

Particular problems with known applicators include that the elongated medical device readily slide through the sheath and can be protruding past the hand while digital insertion occurs. This means the elongated medical device is unprotected and easily deformed or deflected into the wrong direction and can be damaged. Further the end of this elongated medical device, having a small diameter, can become a dangerous and damaging piercing item which could inflict damage to the internal cavity.

It can be seen that the prior art carries some potentially significant flaws including
i) a difficulty with feeding the catheter through the anatomical opening of the external cervical os or the cervical canal;
ii) there can be ready loss of control of device inside vagina
iii) or dropping of small parts inside vaginal canal;
iv) the use of plastic components can cause discomfort or pain to the patient;
v) a risk of damage to the catheter which could affect inflation or cause unintentional deflation;
vi) a considerable change to clinical workflows;
vii) a lack of control of catheter insertion by inexperienced users resulting in damage to the catheter or injuries and discomfort to the patient;
viii) the risk of a foreign body scenario occurring due to the easy detachable nature of the design; and
ix) an increase of medical waste.
It can be seen that known prior art an apparatus and method for guiding or facilitating the insertion of catheters, balloons, and other tubes, wires, rods, threads, or other similar structures into anatomical openings of humans and animals has the problems of:
a) Imprecision in use
b) Amplifies problems for less experienced or less trained users
c) Bulkiness
d) Damage to the medical device due to uncontrolled insertion
e) Injury or discomfort due to uncontrolled insertion
f) Discomfort due to material
g) dislodgment of device
h) awkwardness in use

The present invention seeks to provide an apparatus and method for guiding or facilitating the insertion of catheters, balloons, and other tubes, wires, rods, threads, or similar elongated medical structures into anatomical openings of humans and animals, which will overcome or substantially ameliorate at least one or more of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect of the present invention, there is provided an apparatus for guiding or facilitating the insertion of an elongated medical device into anatomical openings of humans and animals comprising:
a) a hand-controlled body with an operative holding part having at least one of and interacting with the other of:
b) a hand connector connectable with the hand-controlled body and for connecting to the hand that is used for guiding or facilitating the insertion of an elongated medical device and
c) a device connector connectable with the hand-controlled body and for connecting to the medical device that is to be inserted or guiding or facilitating the insertion into anatomical openings of humans and animals
i) the device connector further including a release mechanism
ii) wherein advances the elongated medical device
iii) and disengagement of the medical device in situ
whereby a medical device in use extends along at least one part of the fingers of the user allowing for manual guiding of the elongated medical device into the anatomical opening and further progress and then disengagement allowing retraction of the user's hand.

It can be seen that the invention allows:
(1) Mounting of an elongated medical device directionally along the user's hand;
(2) Partial insertion into or alignment with the anatomical openings of human or animal;
(3) Advancement of the elongated medical device for insertion; and
(4) Release of the medical device from the user's hand to allow the user's hand to be retracted but allowing retention of the medical device in situ.

One form of the invention is for mechanical IOL in humans. In relation to a form of the invention for mechanical IOL, the device is designed to fit within existing clinical workflows whilst also reducing the learning curve and procedural complexities for clinicians to encourage a transition to digital insertions.

It does this by ensuring close contact between the clinician's fingers and the catheter, mounting it to the finger using a wearable sleeve that contains a loop/s or channel within which the catheter is contained. This addresses challenges that are associated with manoeuvring highly flexible, lubricated catheters in intravaginal environment, providing control to the clinician whilst ensuring tactility for guidance. Once the external cervical os is located, to facilitate insertion of the catheter, an actuation or deployment mechanism, with failsafe mechanisms embedded in the device, is activated to gently advance the catheter into the uterus, providing controlled deployment. This addresses the issues associated with generating force and directing said force to advance the catheter into the cervix, as well as limit uncertainties with insertion depth.

There can be different embodiments of the invention and in one form there is provided an apparatus for guiding or facilitating the insertion of an elongated medical device into anatomical openings of humans and animals comprising a hand-controlled body which in use extends along at least part of the fingers of the user, and at least one resistive holding element extending from the hand-controlled body, wherein a medical device can be positioned through the at least one resistive holding element and in use extend along the at least one part of the fingers of the user allowing for guiding the elongated medical device into the anatomical opening.

The hand-controlled body can be a body held by the user's fingers.

The hand-controlled body can be a glove.

The hand-controlled body can be a finger sleeve.

The hand-controlled body can be a ring.

The hand-controlled body can include one or more finger mounted hand-controlled bodies.

The hand-controlled body can have at least one operative holding part which includes at least one or more partially or substantially or completely encircling members through which the elongated medical device can be fed through to ensure its adequate guidance into the anatomical opening. These operative holding parts can be one or more of loops, channels or other tubal structure. In one special form the encircling member is a resiliently closeable C-shape member.

The invention further provides an extension and detent mechanism allowing for deployment by extension of the medical device relative to the device connector to a predefined protruding distance.

The different mechanisms of advancing or actuating the deployment of the elongated medical device can include one or more of:
a) A slide mechanism
b) crossbow/slingshot/elastic launching mechanism
c) a syringe drive mechanism
d) a spring-loaded mechanism
e) a spooling mechanism
f) a pneumatic piston rod driver mechanism
g) a hydraulic piston rod driver mechanism
h) an electromechanical rod driver mechanism
i) a manual or electric rack and pinion mechanism
j) a pulley mechanism and
k) bolt action mechanisms

Failsafe mechanisms in the operative holding part to prevent excessive insertion depths that could be embedded (but not limited to) include: clicks or bumps which can be felt or heard, stoppers (physical/magnetic), visual lines on the device, detents, notches, grooves, protrusions, tabs, ridges, flanges, flaps, gates, flexible members, elongated guide contours, and elongated guide curved surface, reversible locking mechanisms, etc.

The operative holding part can in one form be assembled or disassembled by the user to permit release of the apparatus from the elongated medical device, with the elongated medical device situated in situ.

The operative holding part can in another form be able to be selectively releasable by the user to allow retraction of the hand-controlled body including the operative holding part while retaining the elongated medical device in situ. This can be achieved by at least one operative holding part being selectively releasable by hand and or finger manoeverability such as spreading of fingers to release closing resilience or relative movement of at least part of the fingers of the user along which the hand-controlled body extends.

Preferably the at least one resistive holding element is frangible by one of cutting, causing a weakened location, breaking at a pre-weakened location, or at least partial forcible detachment from the hand-controlled body. Frangibility can include releasing of a hook and loop connector such as Velcro™.

Preferably the apparatus includes an anchoring retention part controllable by the user at a distance from the at least part of the fingers of the user along which the at least one resistive holding element extends along so as to form an anchoring to ensure retraction of the at least one resistive holding element while retaining the elongated medical device in situ. The anchoring retention part can be the remainder of the hand-controlled body, the tight resilient fitting of the hand controlled body on the finger or hand of the user, or by an extended strap extending to be held from a distance from the rest of the operative holding part.

It can be seen that the invention can provide controlled insertion of an elongated medical device. The elongated medical device can include one of:
catheters,
balloons, and
other tubes,
wires,
rods,
threads, or
other similar structures

The invention also provides a method for guiding or facilitating the insertion of catheters, balloons, and other tubes, wires, rods, threads, or other similar structures into anatomical openings of humans and animals for obstetrics and gynaecology including the steps of:
Mounting a hand-controlled body so as to extend along at least part of the fingers of the user
feeding an elongated medical device through one or more at least partially encircling members on the hand-controlled body that will hold the elongated medical device close to the fingers
controlling and manipulating the medical device into the target anatomical opening.
Detaching the encircling member/s to allow the separation of the medical device from the hand-controlled body,
removing of the user's hand or finger(s) and hand-controlled body while retaining the medical device in situ.

It can be seen that the invention provides an apparatus and method for guiding or facilitating the insertion of catheters, balloons, and other tubes, wires, rods, threads, or other similar structures into anatomical openings of humans and animals. This is achieved by feeding the structure through one or more loops on the apparatus that will hold the structure close to the fingers of a suitably trained individual, such as a clinician, to then control and manipulate the tubal structure into the target anatomical opening. The loops can then be detached to allow the separation of the apparatus from the tubal structure, and thus permit removal of the user's hand or finger(s).

In one form the invention includes a glove which has been modified and manufactured in such a way that the catheter can be fed through loops that will help guide the catheter into the cervix, thereby reducing the learning curve of the procedure for clinicians and increasing the rate of insertion.

The invention may provide, in a broad aspect, an apparatus for guiding or facilitating the insertion of catheters, balloons, and other tubes, wires, rods, threads, or other similar structures into anatomical structures of humans and animals. Such anatomical structures include, but are not limited to, the cervix of women via the vaginal route (example procedures: cervical ripening via balloon induction of labour, embryo transfer, balloon tamponade, IUPC insertion, etc.) or the urethra (example procedure: drainage of urine from bladder).

In one embodiment, the device is worn as either a hand glove, available in various sizes based on existing and future sizing practices, or a finger sleeve that is sized in accordance with the various sizes of the index and/or middle fingers of hand gloves; or as a non-customisable one-size fits all.

The device can include at least one or more loops through which the catheter (or other similar device/structure) can be fed through to ensure its adequate guidance to the anatomical opening.

The loops can be manufactured integral onto the gloves and can partially or fully separate from the glove or sleeve through example actions such as tearing or cutting the loop. One such methodology for facilitating this separation is through fenestration of the loops' attachment point(s) to the glove.

In one embodiment, the loops and glove are made from materials that are safe and biocompatible for human use and unlikely to trigger an immune response (such as an allergic reaction). This can be done by using materials that are presently used for the manufacture of medical gloves (such as latex, nitrile, or vinyl) or other suitable material.

Materials used can be Latex, Nitrile, Silicones, Hydrogels, elastomers, Thermoplastics (eg. PLA, PMMA, ABS, Nylon/other polyamides, polyethylene, polypropylene, PPO, PPS, polystyrenes, polyetherimide, PEEK, polycarbonate, PVC, polyetherimide, Polyethersulfone, cyclic olefin copolymer, PGA, PLGA, polyimide, Polyhydroxyalkanoates, Polybutylene succinate, PTFE, Polyvinylidene fluoride or polyvinylidene difluoride, Polydioxanone, polycaprolactone, Polyhydroxybutyrate, Polyhydroxyalkanoates, PDMS), Polymers, Plastics, Metals, Organic materials, Synthetic materials, biopolymers, composites, and biomimetic materials, or combinations as necessary

The device may be provided as is, with an attachable elongated medical device, or with the elongated medical device already pre-loaded. Other necessary items may also be provided as part of a kit.

The device can be supplied in a sterile or non-sterile state and may be supplied in a pre-lubricated or non-lubricated state.

In one embodiment, the device may contain one or more sensors.

In one embodiment, the device may be mounted onto another structure or apparatus that provides access to the anatomical opening, as required by the application.

In one form the invention provides an apparatus and method for guiding or facilitating the insertion of catheters, balloons, and other tubes, wires, rods, threads, or other similar structures into anatomical openings of humans and includes any one or more of the following benefits:
i. improvements in structure and use in order to minimise stress, discomfort, avoidance and trauma by efficient and more effective enabled technique
ii. Improvements in patient comfort;
iii. improvements in clinician skill with procedures requiring insertion of elongated medical devices into anatomical opening;
iv. In increasing patient and provider comfort and experience, it is anticipated that the rate of successful insertion will lead to enhanced patient outcomes
v. apparatus will reduce healthcare waste and expenditure on consumables/equipment that is especially associated with obstetric applications such as IUPC insertion or balloon induction of labour.

Other aspects of the invention as understood from the disclosure of this document by a person skilled in the art are also covered within the scope of the invention.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 is a diagrammatic block diagram of the present workflow in an obstetric procedure using digital insertion technique of a catheter or a speculum insertion technique;

FIG. 2 is a diagrammatic general view of the main elements of an apparatus for use in guiding or facilitating the insertion of an elongated medical device into anatomical openings according to a form of the invention;

FIG. 3 is a schematic drawing of an embodiment of an apparatus for guiding or facilitating the insertion of an elongated medical device into anatomical openings of humans and animals in accordance with the invention in the form of a finger sleeve having a retention member;

FIG. 4, is a schematic drawing of a detail of the hand-controlled body of FIG. 3 showing one form of the device connector having encircling loops;

FIGS. 5 and 6 are schematic drawings of a detail of hand-controlled body such as finger sleeve of FIG. 3 and showing a further embodiment of device connector in the form of a tubular structure holding the elongated medical device;

FIG. 7 is a schematic drawing from a cross sectional view of the fingers of a further embodiment of an apparatus for guiding or facilitating the insertion of an elongated medical device into anatomical openings of humans and animals in accordance with the invention with the hand connector in the form of a finger hold apparatus that also provides a device connector in a clamping form;

FIGS. 8 to 18 are diagrammatic view of various embodiments of extension and detent mechanism for use in the apparatus for guiding or facilitating the insertion of an elongated medical device, including one or more controlled extension mechanisms of the medical device to a predetermined distance comprising of a slide mechanism, crossbow/ slingshot/elastic launching mechanism, a spring-loaded mechanism, a spooling mechanism, a pneumatic piston rod driver mechanism, a hydraulic piston rod driver mechanism, a manual rack and pinion mechanism, a pulley mechanism and bolt action mechanisms;

FIGS. 19 and 20 are the parts and the assembly of the parts in a particularly preferred embodiment of an apparatus for guiding or facilitating the insertion of an elongated medical device using a sliding mechanism as extension and detent mechanism;

FIG. 21 is a cross sectional view of a detail of FIG. 20 showing the inclusion of stop mechanisms in the device connector to engage the sliding mechanism and limit advancement of the medical device to the predetermined distance;

FIGS. 22 and 23 are diagrammatic views of the movable engagement of device connector to the hand connector at a point which allows rotational movement and/or angular movement of the medical device relative to the operator's guiding hand or finger;

FIG. 24 is a diagrammatic view of the steps of the obstetric procedure that an embodiment of the apparatus for use in guiding or facilitating the insertion of an elongated medical device can be used;

FIGS. 25 and 26 are schematic block step diagrams of a general and more specific method for guiding or facilitating the insertion of catheters, balloons, and other tubes, wires, rods, threads, or other similar structures into anatomical openings of humans and animals for obstetrics and gynaecology.

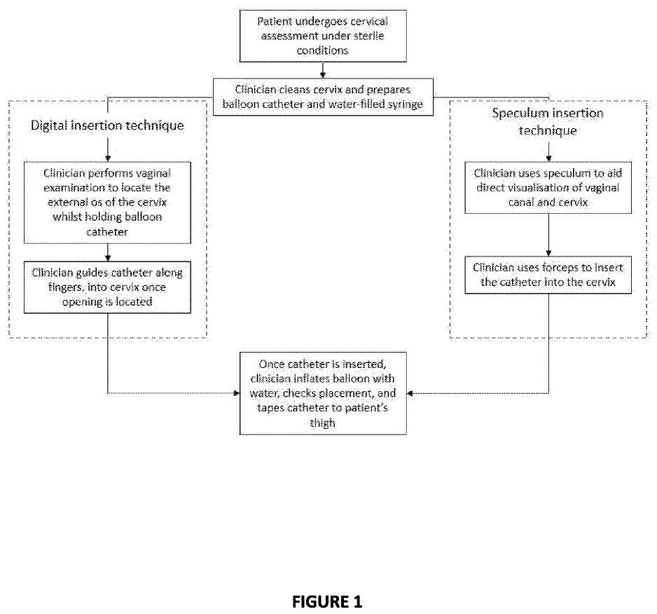

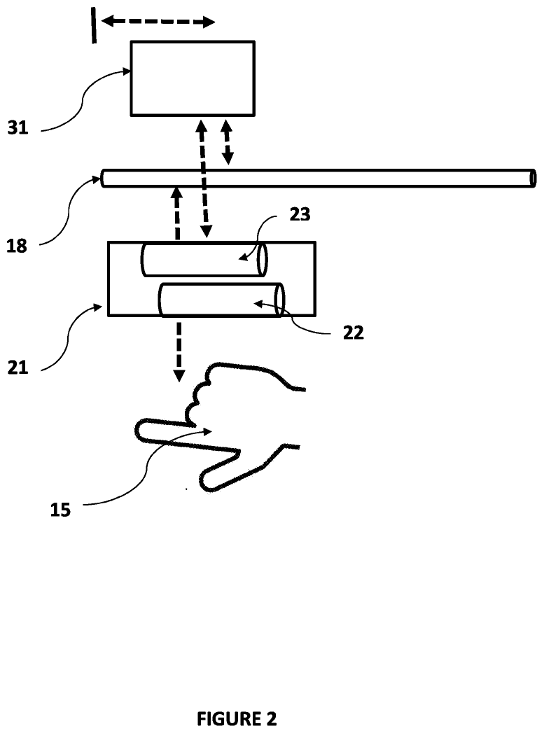

### DESCRIPTION OF PREFERRED EMBODIMENTS

It should be noted in the following description that like or the same reference numerals in different embodiments denote the same or similar features.

Referring to the drawings there is shown an apparatus for use in obstetrics and gynaecology to simplify and improve guidance of the insertion of an elongated medical device into anatomical openings of humans and animals.

The device is in one form an apparatus for guiding or facilitating the insertion of an elongated medical device 18 into anatomical openings of humans and animals comprising a hand-controlled body 21 with a hand connector 22 and a device connector 23 connectable therewith. The hand-controlled body 21 is for connecting to the hand that is used for guiding or facilitating the insertion of an elongated medical device 18 that is to be guiding or facilitating the insertion into anatomical openings of humans and animals.

The hand-controlled body 21 has an extension and detent mechanism 31 allowing for deployment by extension of the medical device 18 relative to the device connector to a predetermined distance. In this way the medical device can be advanced, at the required time, to protrude past the hand of the operator and safely further extend into the anatomical opening of the humans and animal patient or part thereof.

The device connector 23 can further include a release mechanism 41 wherein allowing further progress of the medical device and disengagement of the medical device in situ and whereby a medical device in use extends along at least one part of the fingers of the user allowing for manual guiding of the elongated medical device into the anatomical opening and further progress and then disengagement allowing retraction of the user's hand.

The apparatus can comprise a hand-controlled body 21 which in use extends over the hand 15 and fingers 16 of the user. In a first embodiment of a glove an operative holding part 22 mounted on the index finger of the user and connected with the hand-controlled body 21 and for holding part of an elongated medical device 18 along the hand. Clearly the operative holding part 22 can be mounted on other fingers of the user and particularly preferable is mounting on the middle finger as it protrudes the furthest and is quite manouverable and able to co-work with the thumb.

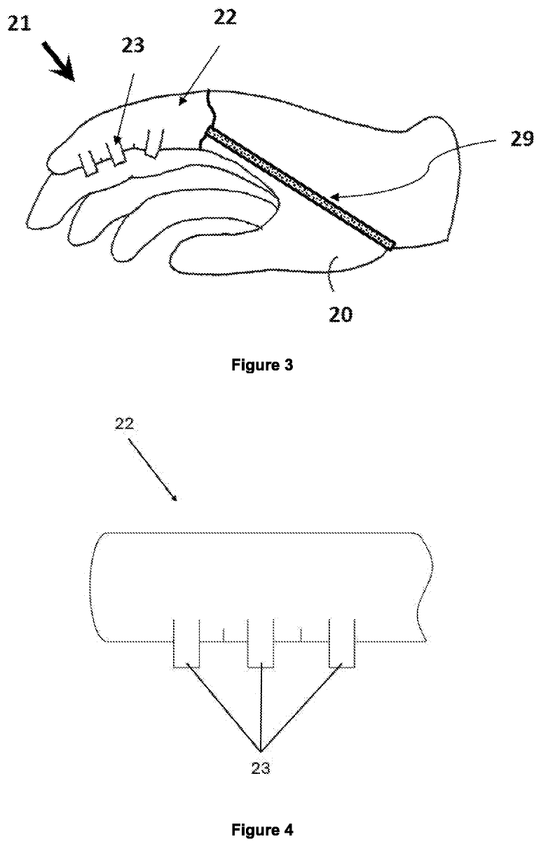

The operative holding part or device connector 23 can include multiple parts extending from hand-controlled body 21 and even partially or fully encircling the finger of the user. As shown in FIGS. 3 and 4 there are three loops forming the operative holding part of the device connector 23 and able to hold an elongated medical device 18 between the loops and the index finger of the user. In this way the medical device 18 in use extends along the index finger of the user allowing for manual guiding of the elongated medical device into the anatomical opening.

The hand-controlled body 21 can include an anchoring retention part 29 for aiding retention of the operative holding part on at least part of the hand or fingers of the user. When the hand-controlled body 21 is a glove then the remainder of the glove forms the anchoring retention part 29 and particularly fits around the user and so allowing the fingers to be inserted and extricated without losing the glove.

As detailed in FIGS. 3 and 4, when the hand-controlled body 22 is a finger sleeve, the anchoring retention part extends from the finger sleeve and held by at least one finger of the user or the remainder of the user's hand. The anchoring retention part 29 when in the form of a strap can be controllable by the user at a distance from the at least part of the fingers of the user along which the operative holding part extends along so as to form an anchoring to ensure retraction of the at least one resistive holding element while retaining the elongated medical device in situ.

The hand-controlled body can include a finger ring (not shown) wherein one or more parts of the at least one holding element is mountable to encircle the finger of the user.

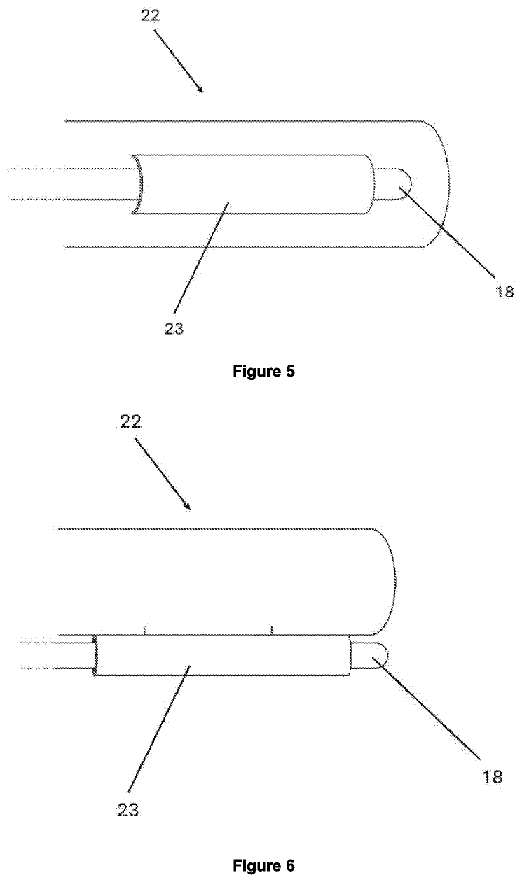

As shown in FIGS. 5 and 6 the hand-controlled body 21 such as finger sleeve of FIGS. 3 and 4 can include a further embodiment of device connector 23 in the form of a tubular structure holding the elongated medical device 18.

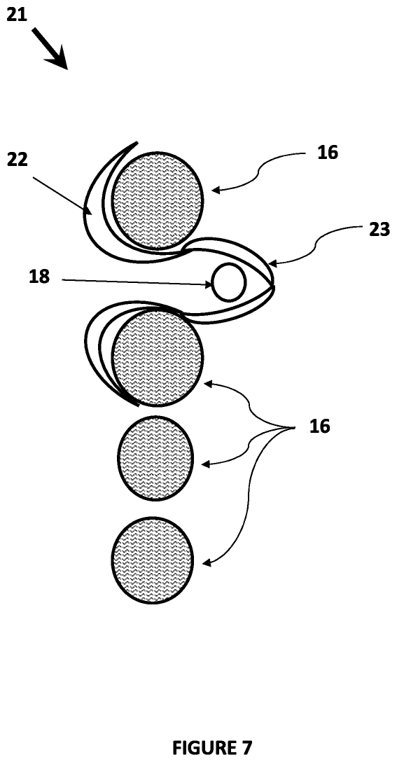

As detailed in FIG. 7 the hand-controlled body 21 is a finger hold and can include an operative holding part of device connector 23 is in the form of a finger loop extending outwardly from between two adjacent fingers of the index finger and the middle finger and substantially encircling the elongated medical device 18 therebetween. The anchoring retention part 29 can be considered to be a W shape finger loop base that extends around the back of the index finger and the middle finger holding the elongated medical device 18 within the finger loop. Therefore, the hand-controlled body 21 is controllable by the user by at least part of the fingers of the user along which the operative holding part extends along so as to form an anchoring to ensure retraction of the at least one resistive holding element while retaining the elongated medical device in situ.

The operative holding part is able to be selectively releasable by the user to allow retraction of the hand-controlled body including the operative holding part while retaining the elongated medical device in situ. This can be achieved by at least one operative holding part being selectively releasable by hand and or finger manoeverability such as spreading of fingers to release closing resilience or relative movement of at least part of the fingers of the user along which the hand-controlled body extends.

Preferably the at least one resistive holding element is frangible by one of cutting, breaking at a pre-weakened location or at least partial forcible detachment from the hand-controlled body. Frangibility can include releasing of a hook and loop connector such as Velcro™.

It can be seen that in one form the present invention is a medical hand glove or finger sleeve for guiding, facilitating the control, or manipulation of catheters, balloons, and other tubes, wires, rods, threads, or other similar structures into anatomical openings of humans and animals. It is intended that the hand glove is worn on the hand before use, whilst the finger sleeve (a separate but related apparatus) is worn on the finger/s. The apparatus and/or elongated medical device (henceforth referred to as a catheter as an example), is then lubricated at the loops to permit easy feeding of the catheter through the loops and reduce friction that is associated with the glove material. Typically, catheters are lubricated before insertion into the body, so this practice does not require anything extra beyond the clinical standard. Pain medication such as lidocaine gel or other clinically accepted analgesic may be applied as required. The catheter is then fed through the loops.

### Extension and Detent Mechanism (for Deployment)

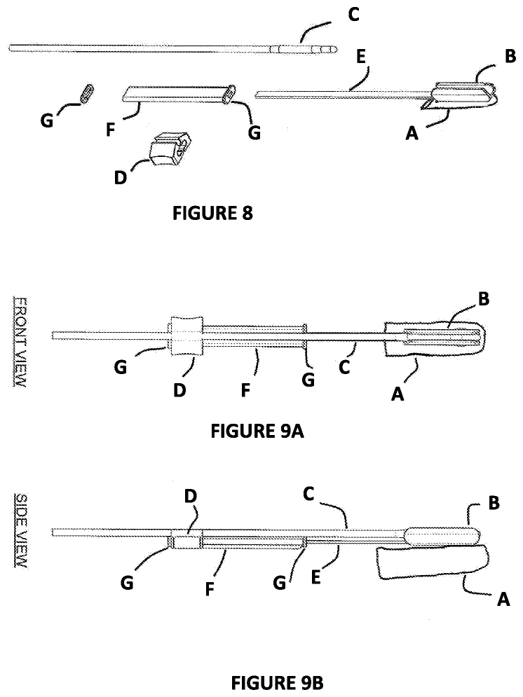

Referring to FIGS. 8 to 18 there are shown alternative deployment mechanisms of the extension and detent mechanisms 31 which can be one of or a combination of the below mechanisms.

The examples below illustrate scenarios generally where the catheter carrier (containing the catheter) is the primary moving component of the device, often along a fixed rail and through a fixed finger sleeve and loop assembly. However, it is possible to envisage scenarios wherein the catheter carrier is fixed to the fixed finger sleeve and loop assembly, and the rail, using the same actuation mechanism principles, becomes a movable platform that can advance the catheter.

The features of these examples can be identified as:
a) A: Finger sleeve—hand connector 22
b) B: Loop/carrier/channel—device connector 23
c) C: Catheter—medical device 18
d) D: Catheter carrier which slides along rail or loop (B)—extent and detent mechanism 31
e) E: Region between loop (B) and rail (F)
f) F: Rail for sliding mechanism or directing action
g) G: Stop mechanisms to control distance and limit sliding mechanism
h) H: Stop similar to (G) but serves as limb for attaching string (I) to
i) I: Elastic band or string under tension
j) J: Trigger to release string and launch catheter forward
k) K: Spring
l) L: Rod to drive catheter carrier (D)
m) M: Chamber 2 (valve not shown)
n) N: Bore or piston
o) O: Chamber 1 (valve not shown)
p) P: Spool turns string around limb (H) to draw catheter carrier (D) forward
q) Q: Rack
r) R: Pinion with shaft for manual rotation using winding action
s) S: Stop (G) with a hole for string (I) to pass through
t) T: Tab to be pulled to trigger motion
u) U: Bolt handle attached to catheter carrier (D)
v) V: Slot to guide bolt (U) for sliding catheter carrier (D)

FIGS. 8 and 9A and 9B mechanism is a Manual Sliding Mechanism where the catheter is loaded in catheter carrier (D) and in (B). D then slides forward along Rail (F) between points G to advance the catheter into the cervix. After this, the catheter is inflated and the entire unit slides along the catheter where it can be removed.

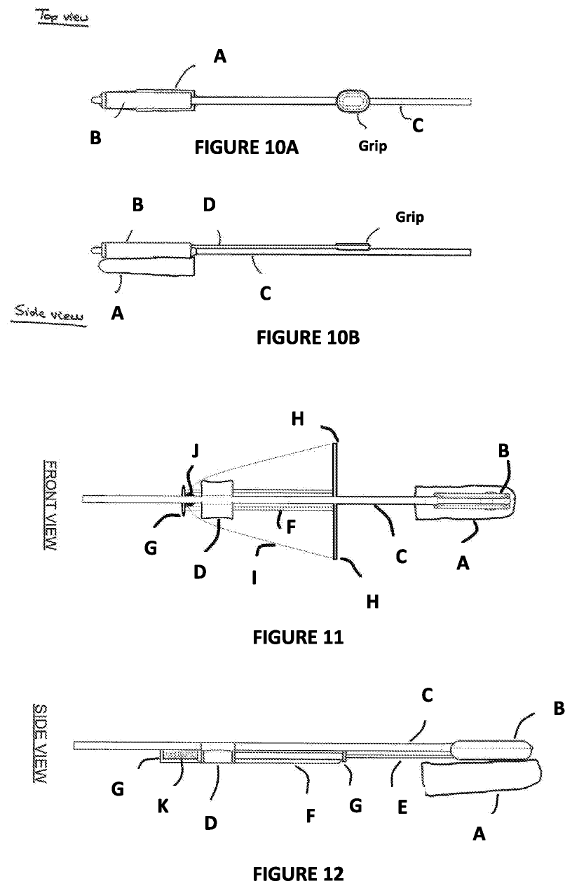

FIGS. 10A and 10B mechanism is similar to the manual sliding mechanism of FIG. 8, except that the catheter carrier (D) slides along the loop/carrier (B), and (D) may have a shaft which the clinician uses to advance the catheter. A rail (F) may be embedded in (B).

FIG. 11 is a crossbow/slingshot/elastic launching mechanism in which the catheter is loaded in catheter carrier (D) and in (B). D is then pulled back along Rail (F) to the latch/trigger (J) for securing. Notable components are the string (I) which is used to launch the catheter when elastic potential energy is released using J, and the limbs (H) which the string is anchored on. Once the cervical opening is located, the clinician can release the trigger and launch the catheter carrier (D) to advance the catheter into the cervix. After this, the catheter is inflated and the entire unit slides along the catheter where it can be removed.

A mechanism not illustrated is a syringe drive mechanism in which (D) is connected to the screw mechanism of the driver which gently drives (D) forward and into the cervix when activated. Alternatively, (D) is driven forward by an action that is in conjunction with the driver (if using Sliding Mechanism 1).

Referring to FIG. 12 there is a spring-loaded mechanism which is similar to the crossbow mechanism and the manual sliding mechanism 1. Catheter is loaded in catheter carrier (D) and in (B). Once the cervical opening is located, D is then pulled back along Rail (F) to compress the Spring (K) and store elastic potential energy. Upon release, this stored energy is converted into kinetic energy that launches the catheter forwards and into the cervix. After this, the catheter is inflated and the entire unit slides along the catheter where it can be removed.

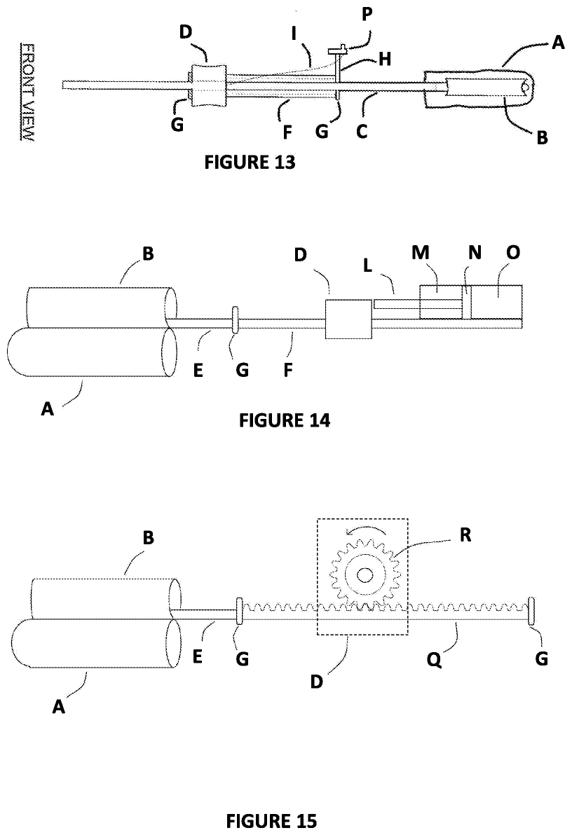

FIG. 13 is a spooling mechanism. In this mechanism, a string (I) is connected to the catheter carrier D. Catheter is loaded in catheter carrier (D) and in (B). Once the cervical opening is located, spool (P) is rotated such that the string starts coiling around limb (H) and draws the catheter carrier (D) forward and the catheter enters the cervix. After this, the catheter is inflated and the entire unit slides along the catheter where it can be removed.

Referring to FIG. 14 there is a pneumatic piston rod driver mechanism. This example uses a double acting pneumatic system. Valves not shown. The catheter is loaded in catheter carrier (D) and in (B). Once the cervical opening is located, clinician allows air or a gas (compressed or non-compressed) to enter Chamber 1 (O) and air to exit Chamber 2 (M) which forces the piston (N) to advance rod (L) forward. This action of rod L pushes the catheter carrier (D) forward, allowing catheter to enter the cervix. After this, the catheter is inflated and the entire unit slides along the catheter where it can be removed. NOTE: This mechanism could work with a hydraulic piston system where liquids (instead of gas) such as water may enter Chamber 1 (O) to drive the piston forward.

FIG. 15 shows a rack and pinion mechanism. A catheter is loaded in catheter carrier (D) and in (B). Once the cervical opening is located, clinician activates pinion (R) by scrolling or winding the mechanism (if a shaft is provided) to shift the catheter carrier (D) along the rack (Q) such that the catheter advances into the cervix. After this, the catheter is inflated and the entire unit slides along the catheter where it can be removed. This mechanism can be electromechanically controlled as well if the pinion is connected to a DC motor.

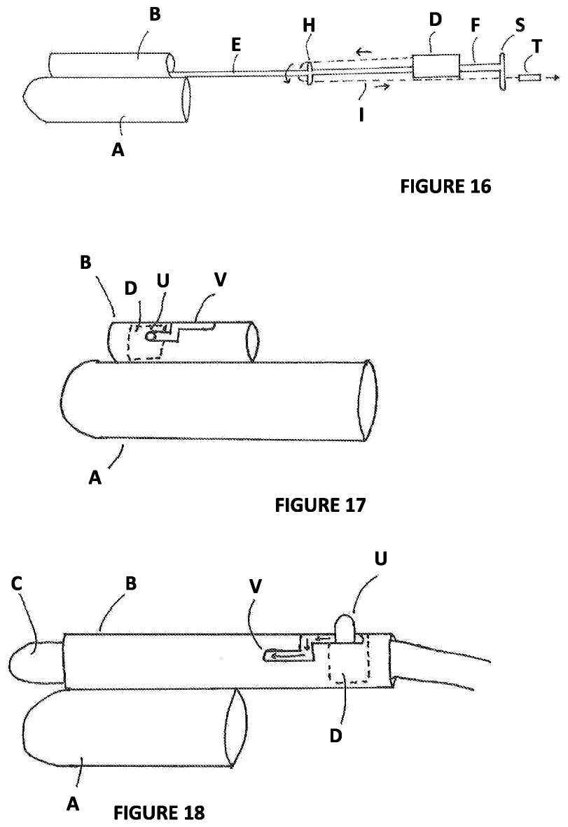

Referring to FIG. 16 there is a pulley mechanism in which a string (I) is connected to the catheter carrier D, passes over limb (H), and proceeds along the undercarriage of the device to pass through S and connect to tab T. First, the Catheter is loaded in catheter carrier (D) and in (B). Once the cervical opening is located, the tab (T) is pulled by the clinician causing D to move forward and drive the catheter into the cervix. After this, the catheter is inflated and the entire unit slides along the catheter where it can be removed.

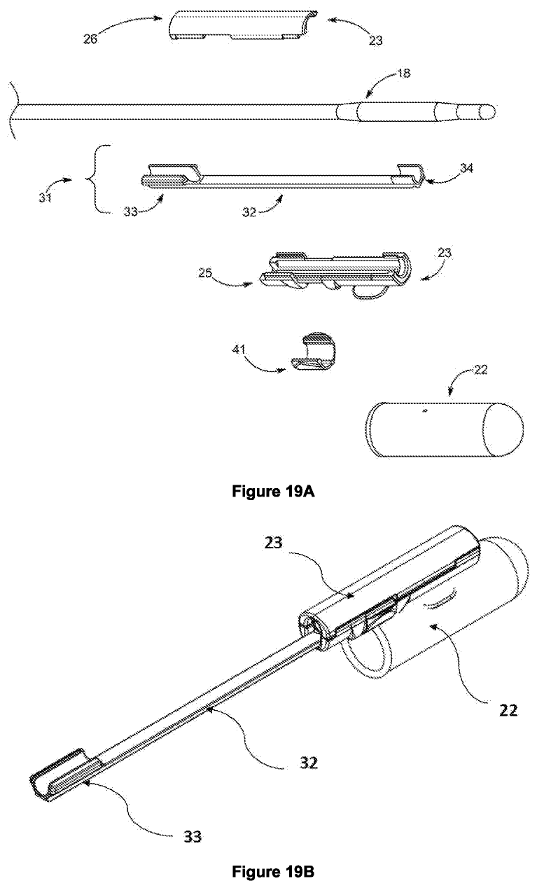

FIGS. 17 and 18 have bolt actions which draws upon similarities with the manual sliding mechanism 2, where D goes inside B and is moved to advance the catheter; in this design, D is connected to a bolt (U) which protrudes from and slides along gap V. This allows the clinician to employ a bolt action style mechanism to deploy the catheter.

A lateral (or along an arc) aspect of the gap allows D to rotate within B to control depth of insertion whilst maintaining the same position in 3-dimensional space, especially when following the cylindrical coordinate system that would apply with the shape of B. After this, the catheter is inflated in the cervix and the entire unit slides along the catheter where it can be removed. Arc refers to arc of a circle as would apply in a cylindrical style loop (B). Designs for gap V that are similar to manual gearshift can be considered.

### Particular Preferred Embodiment

Referring to FIGS. 19A and 19B, 20 and 21 there is shown an embodiment of the apparatus for guiding or facilitating the insertion of an elongated medical device into anatomical openings of humans and animals. The whole device or apparatus of this embodiment involves components of:
A) the catheter 18 forms the medical device
b) The device connector 23 forming part of the hand-controlled body 21 is to substantially capture the catheter 18.
c) The hand connector 22 connected to the device connector 23 forms the other part of the hand-controlled body 21; and
d) The slide 31 forms the slide mechanism that is in this embodiment the extension and detent mechanism allowing for deployment by extension of the medical device relative to the device connector to a predefined protruding distance.
e) The release mechanism 41 for allowing release of the catheter from the device connector to allow removal of the device connector and operator hand while retaining the catheter in situ.

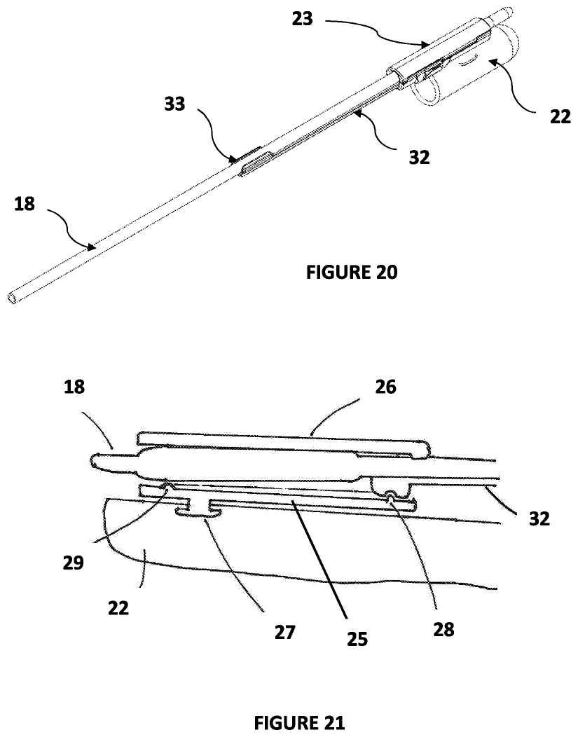

The parts of the hand-controlled body 21, which holds the medical device 18 of the balloon catheter in this procedure, are shown in exploded view in FIG. 19A and in assembled form in FIG. 19B and in use in FIG. 20 and in cross-sectional detail in FIG. 21.

The parts comprise a hand connector 22 in the form of a finger sleeve to be mounted on the operator's finger and a connected device connector 23. The connection between the two can be an interfacing connector 27 extending from one to the other to form a movably connected arrangement so that digital control of the hand connector allows control of the device connector.

The device connector 23 includes open capturing channel guide base 25 with an interfitting guide top cover 26 which clips onto open capturing channel and conceals the inner shapings to avoid any interaction when inserted into anatomical opening. A release mechanism 41 (in this example, tabs, which when pinched, release the top cover 26. These tabs may also help lock the top cover 26 onto the base 25.

The extension and detent mechanism 31 includes a sliding mechanism of an open channel shaft 32 with a clip 34 at one end to that supports the medical device and slide within the device connector 25 and a grip 33 at the other end for the operator to initiate the slide and advance the medical device 18 at the required time to protrude out of the device connector the predetermined distance.

As shown in the detail of FIG. 21 the inside of the device connector 23 includes a bump lock 28 at one internal end for the start extent of the medical device while digital insertion and the bump extent 29 at the other internal end of the device connector that the slide 32 encounters when the operator uses the grip 33 and to advances the slide 32 and mounted catheter 18 to the predetermined protruding distance.

The hand connector is shaped and sized for connecting and mounting to the hand that is used for guiding or facilitating the insertion of an elongated medical device and the device connector is shaped and sized for capturing the medical device that is to be guiding or facilitating the insertion into anatomical openings of humans and animals. This allows the medical device in use to extend along at least part of the hand connector and along one finger of the user allowing for manual guiding of the elongated medical device into the anatomical opening.

The apparatus further includes a release mechanism to allow disengagement of the medical device in situ and allowing retraction of the user's hand and the hand-controlled body while allowing retention of the medical device in situ.

It can be seen that the invention includes, in one form, three main concepts.
a) The finger sleeve or hand connector provides clinicians with a tactile means of interaction with the cervix. Through its interface with the device connector, it enables precise handling and control of the catheter. This ensures dexterity and maneuverability of the catheter.
b) The catheter carrier holds the catheter inside the operative holding part or device connector and permits advancement of the catheter through the device connector and into the cervical canal via a directionally applied force that slides the catheter forward. This forms part of the extension and detent mechanism and permits controlled catheter advancement.
c) The device connector provides stability and enables controlled directional actuation of the catheter into the cervical canal. In this example, an open capturing channel is loaded with the catheter carrier and catheter. The top cover clips on with the aid of the release mechanism; thereby enclosing the operative holding part and completing the hand-controlled body. This improves controllability of the catheter and helps direct the catheters advancement into the cervix. Failsafe mechanisms are further embedded in this part 28, 29.

A step is to have the catheter advance further into the target anatomical structure (cervical canal or uterus). In this crucial step, the device ensures the correct positioning of the catheter within the cervical region, ready for advancement. The device securely holds the catheter, facilitating the controlled progression of the catheter by the clinician using their other hand. This step underscores the process of ‘loading the catheter to a hand-controlled body’.

By engaging the side buttons or tabs 41 (or other release mechanism) integrated into the guide's open capturing channel and top cover of parts 26 and 27 of the device connector 23, clinicians effortlessly unclips the top cover 26 from the device connector and open access to the catheter 18. This mechanism ensures swift and secure disassembly, a vital feature for enhancing device's usability and efficiency.

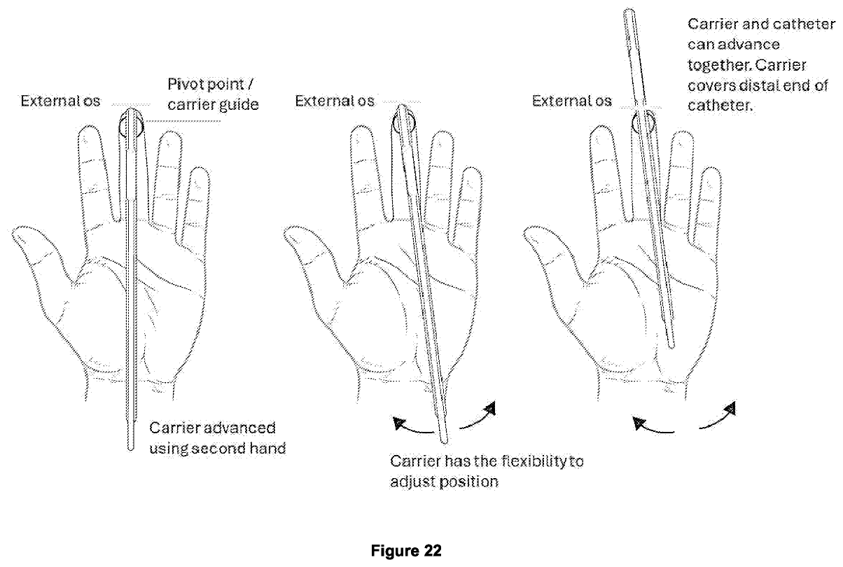

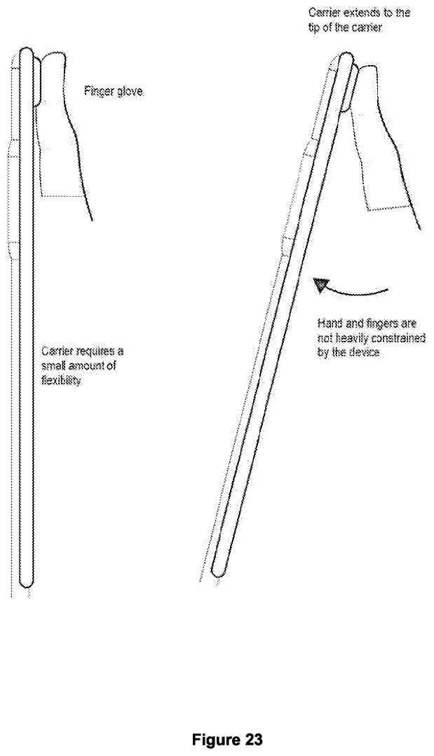

Referring to FIGS. 22 and 23 there is shown an embodiment of the apparatus wherein the hand connector covers the at least one of the fingertips of the fingers of the user to provide the connecting to the hand. The device connector is movably engaging one point of the hand connector allowing movement of the device connector relative to the hand connector around the one point.

This provides a mechanism in which the device connector can exhibit a swivel capability or it may be fixed. The unique feature of articular movements provides clinicians with the ability to flexibly adjust its position, allowing the device connector to manoeuvre around a point, eliminating the excessive needs for finger, hand, or wrist adjustments. This reduces the risk of injury to the clinician and patient discomfort while accommodating individual anatomical variations.

### Usage

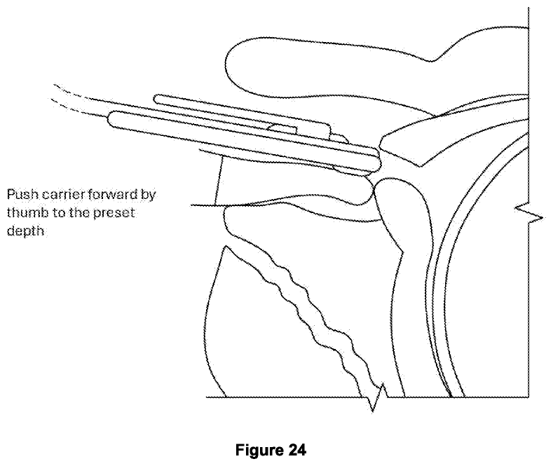

Referring to FIG. 24 there is shown an application of usage in an obstetric procedure. In Step 1 the balloon of the balloon catheter is in the device connector with the catheter extending along the hand of the operator and across the wrist. The balloon is not inflated and within the device connector which due to its connection to the hand connector in the form of a finger sleeve is mounted on the digit of the operator but does not protrude beyond the fingertip of the operator. The mounting of the hand connector on the operator and the non-protrusion, by the relative connected position of the device connector, allows the operator to safely and with control locate the medical device at the required location. It is at this stage at Step 2 that after locating the operator advances fingertip to the external cervical OS.

However, at Step 3 the operator initiates the deployment mechanism of the extension and detent mechanism to extend the balloon catheter out of the device connector to a predetermined distance.

At Step 4 the balloon is inflated at the required position, the hand-controlled body is withdrawn along the catheter out of the vagina, and by release mechanism the catheter can be released from the device connector and allow the whole hand-controlled body including the device connector and the hand connector and the hand of the operator be removed while retaining the balloon catheter in situ in operative form.

In use, once the catheter and loops are sufficiently lubricated, the finger is aligned with the anatomical opening (such as a cervix which requires access through the vagina) and the finger and loops guide the insertion of the catheter into the anatomical opening. Once inside the opening, the catheter can then be deployed and function as intended. In the example of cervical ripening which employs a balloon catheter, the balloon can be inflated to induce labour.

When extracting the user's digits or hand, the catheter can be held in place whilst the loops permit a sliding action down the catheter to a point where it is safe to cut, tear, disassemble or otherwise detach the loop/s and permit removal of the hand, digit, and apparatus from the patient's body. For some designs, the loops need not be separated and the device may remain connected to the catheter outside of the body, or slide off the catheter as a whole (i.e. without detaching the loops).

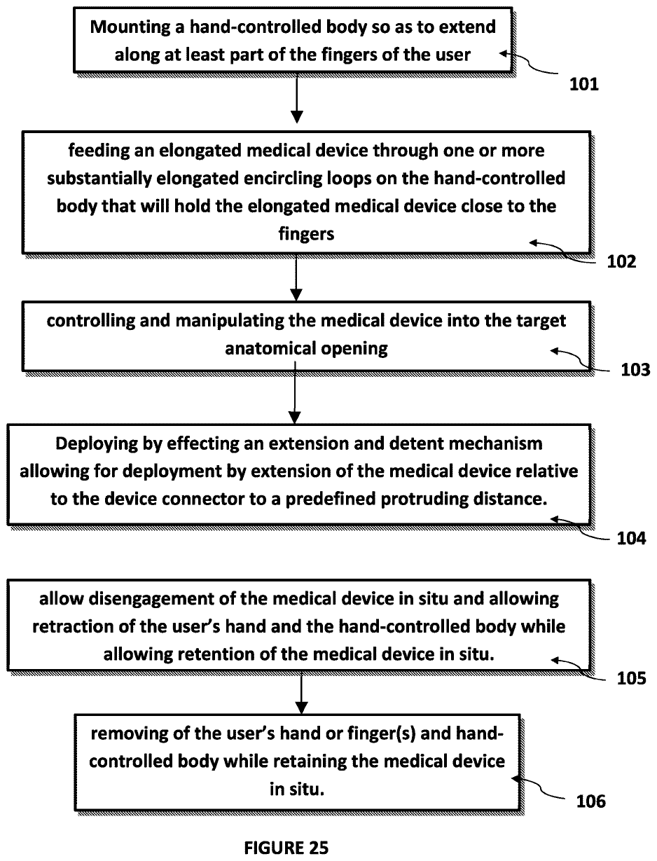

Therefore, as shown in FIG. 25 the steps in use comprise of step 101 of mounting a hand-controlled body so as to extend along at least part of the fingers of the user. This can be a glove 11 over a hand 20 or a finger sleeve 31 over a finger 12.

Step 102 comprises feeding an elongated medical device 18 through one or more operative holding parts on the hand-controlled body 21 that will hold the elongated medical device 18 close to the fingers 16. The user is therefore in step 103 able to control and manipulate the medical device 18 into the target anatomical opening.

Step 104 is undertaking deployment of the medical device by effecting an extension and detent mechanism. This provides an extension of the medical device relative to the device connector to a predefined protruding distance.

In step 105 when the medical device 18 is to remain in situ, the user uses a releasing mechanism to disengage the medical device from the device connector of the hand-controlled body such as detaches the loops and to allow the separation of the medical device from the hand-controlled body. This allows step 106 of removing of the user's hand or finger(s) and hand-controlled body while retaining the medical device in situ.

Although the present invention has been described with reference to preferred embodiments, workers skilled in the art will recognize that changes may be made in form and detail without departing from the spirit and scope of the invention. This includes, and is not limited to, concepts such as loops that go over one or more fingers in any way, gloves or finger rod sleeves without fingertips, rings, bands, tapes, constructs of any length and shape, straps of any kind (including adjustable or detachable and reattachable), incomplete loop structures (similar to hooks), or any other methods of creating a loop-like interface through which another object can go through. It is also recognized that the device be applied to applications beyond obstetrics and gynaecology. Lastly, it is recognized that other actuation mechanisms may be considered relevant.

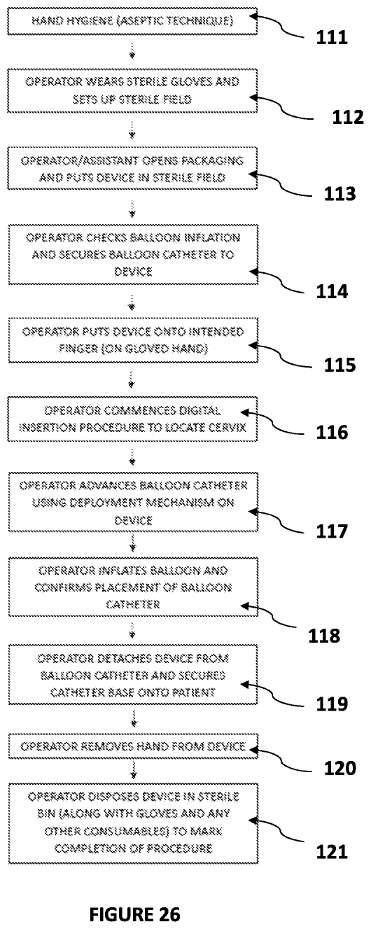

In FIG. 26 the steps of operation in order to achieve the obstetric procedure of mechanical IOL of FIG. 24 with the apparatus of the invention includes the step 111 of undertaking hand hygiene by aseptic washing and then in step 112 operator wears sterile gloves and sets up a sterile field for the procedure. In step 113 the operator opens the packaging that retains the device in sterile condition and places the sterile device in the sterile field.

A check step 114 follows in which the operator checks the balloon associated with the catheter inflates and secures balloon catheter to the device or apparatus of the invention for guiding or facilitating the insertion of an elongated medical device into anatomical openings of humans and animals.

In step 115 the operator puts the hand connector part of the device onto intended finger (if it is a fingertip form of hand connector) which is over the gloved hand of the operator. The glove being a sterile surgical glove.

In step 116 the operator commences digital insertion procedure to locate the cervix and in step 117 advances balloon catheter to general location then using the deployment mechanism to further extend to predetermined distance. Now at the required location in step 118 the operator inflates the balloon with water through the catheter to the balloon which now released from the extension and detent mechanism allows ready expansion.

With the inflated balloon in position in step 119 the operator withdraws from the vagina and then detaches the device from the balloon catheter by way of the release mechanism that allows the catheter to be withdrawn through an openable or open section of the device connector.

### Interpretation

### EMBODIMENTS

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Preferred Embodiments are hereby expressly incorporated into this Detailed Description of Preferred Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are particularly applicable to the obstetrics and gynaecology industries. However they also apply to other medical, veterinary, forensic, scientific and research fields and other healthcare industries that treats or aids or reviews humans and animals, or parts thereof.

1-48. (canceled)

49. An apparatus for use in guiding or facilitating the insertion of an elongated medical device into anatomical openings of humans and animals, comprising:
a hand-controlled body having:
a) a hand connector shaped and sized for connecting and mounting to the hand that is used for guiding or facilitating the insertion of an elongated medical device; and
b) a device connector shaped and sized for capturing the medical device that is to be guided into the anatomical opening of humans and animals;
wherein a medical device in use extends along at least part of the hand connector and along at least one finger of the user allowing for manual guiding of the elongated medical device into the anatomical opening the apparatus further including:
c) an extension and detent mechanism allowing for deployment by extension of the medical device relative to the device connector to a predetermined distance.

50. The apparatus according to claim 49, further comprising:
a release mechanism to allow disengagement of the medical device in situ and allowing retraction of the user's hand and the hand-controlled body while allowing retention of the medical device in situ.

51. The apparatus according to claim 50, wherein the hand connector covers the at least one part of the fingers of the user to provide the connection to the hand.

52. The apparatus according to claim 50, wherein the hand connector resiliently engages between at least one part of the fingers of the user to provide the connection to the hand.

53. The apparatus according to claim 50, wherein the device connector is movably engaged with at least one point of the hand connector allowing movement of the device connector relative to the hand connector around the at least one point.

54. The apparatus according to claim 50, wherein the device connector includes an open capturing channel for releasably capturing at least part of the medical device and further includes one or more of:
a. clips for releasably capturing at least part of the medical device;
b. a sleeve for connection to the medical device; and
c. a resilient clamp for clamping the medical device.

55. The apparatus according to claim 54, wherein the device connector includes a stop mechanism to control distance of the extension and detent mechanism allowing for deployment by extension of the medical device relative to the device connector to a predetermined distance.

56. The apparatus according to claim 54, wherein the device connector includes a sliding mechanism to move the medical device and includes a stop mechanism to limit the sliding mechanism to effect the movement to the predetermined distance.

57. The apparatus according to claim 50, wherein the open capturing channel is closed by a removable cover and the release mechanism allows removal of the closed channel and removal of the medical device from the device connector to allow the medical device to remain in situ while the device connector is removed.

58. The apparatus according to claim 57, wherein the removable cover is one of:
d. a frangible cover;
e. a sliding cover;
f. a tear off strip; and
g. a releasable cover that is released by movement of one or more releasable tabs.

59. The apparatus according to claim 49, wherein the extension and detent mechanism includes one or more mechanisms of:
h. a slide mechanism;
i. a crossbow/slingshot/elastic launching mechanism;
j. a syringe drive mechanism;
k. a spring-loaded mechanism;

1. a spooling mechanism;
m. a pneumatic piston rod driver mechanism;
n. a hydraulic piston rod driver mechanism;
o. an electromechanical rod driver mechanism;
p. a manual or electric rack and pinion mechanism;
q. a pulley mechanism; and
r. bolt action mechanisms.

60. The apparatus of claim 49, wherein the device connector includes an operative holding part for holding part of the elongate medical device, the operative holding part being connectable with the hand connector and adapted to be selectively releasable by hand or finger maneuverability by relative movement of at least part of the fingers of the user along which the hand-controlled body extends.

61. The apparatus of claim 49, wherein the hand-controlled body includes an anchoring retention part for aiding retention of the operative holding part on at least part of the hand or fingers of the user.

62. The apparatus of claim 61, wherein the hand-controlled body is a finger sleeve mountable on at least one finger of the user, and wherein the anchoring retention part extends from the finger sleeve and held by at least one finger of the user or the remainder of the user's hand.

63. The apparatus according to according to claim 61, further comprising an extension and detent mechanism including a stop mechanisms controlling insertion depth and allowing for deployment by extension of the medical device relative to the device connector to a predetermined distance.

64. The apparatus according to claim 61, wherein the operative holding part includes at least one resistive holding element which is selectively releasable by the user to allow retraction of the hand-controlled body and the at least one resistive holding element while retaining the elongated medical device in situ.

65. An apparatus for use in guiding or facilitating insertion of an obstetric or gynecological device via the cervix of women via the vaginal route, comprising:
a hand-controlled body having:
s. a hand connector shaped and sized for connecting and mounting to the hand that is used for guiding or facilitating the insertion of an elongated medical device; and
t. a device connector shaped and sized for capturing the medical device that is to be guided into the vaginal opening of the female subject, the device connector including an operative holding part for holding part of the elongate medical device, the operative holding part being connectable with the hand connector; and
u. an anchoring retention part for aiding retention of the operative holding part on at least part of the hand or fingers of the user;
wherein a medical device in use extends along at least part of the hand connector and along at least one finger of the user allowing for manual guiding of the elongated medical device into the anatomical opening;
the apparatus further including:
v. an extension and detent mechanism allowing for deployment by extension of the medical device relative to the device connector to a predetermined distance; and
w. a release mechanism to allow disengagement of the medical device in situ and allowing retraction of the user's hand and the hand-controlled body while allowing retention of the medical device in situ.

66. The apparatus according to claim 65, wherein the operative holding part is frangible by one of:
x. application of force;
y. cutting;
z. chemically or heat creating a weakened location;
aa. breaking at a pre-weakened location; and
bb. at least partial forcible detachment from the hand-controlled body.

67. The apparatus according to claim 65, wherein the predefined procedures includes via:
cc. the cervix of women via the vaginal route:
i. mechanical induction of labour (IOL);
ii. fetal scalp electrode (FSE) insertion;
iii. embryo transfer during in-vitro fertilisation (IVF);
iv. Bakri balloon/balloon tamponade insertion for PPH;
v. hystero-contrast-sonography (HyCoSy) or hysterosalpingography (HSG);
vi. intrauterine pressure catheter (IUPC) insertion;
vii. other transcervical procedures;
viii. intrauterine insemination (IUI);
ix. cervical screening; and
x. any other procedure seeking uterine access via the cervix;
dd. the urethra:
i. drainage of urine from bladder; and
ii. urinary catheterization.

68. A method for guiding or facilitating the insertion of catheters, balloons, and other tubes, wires, rods, threads, or other similar structures into anatomical openings of humans and animals for obstetrics and gynecology, including the steps of:
ee. mounting a hand-controlled body so as to extend along at least part of the fingers of the user;
ff. feeding an elongated medical device through one or more substantially encircling loops on the hand-controlled body that will hold the elongated medical device close to the fingers;
gg. controlling and manipulating the medical device into the target anatomical opening;
hh. deploying by effecting an extension and detent mechanism allowing for deployment by extension of the medical device relative to the device connector to a predefined protruding distance;
ii. allow disengagement of the medical device in situ and allowing retraction of the user's hand and the hand-controlled body while allowing retention of the medical device in situ; and
jj. removing of the user's hand or finger(s) and hand-controlled body while retaining the medical device in situ wherein the medical device can include one of:
i. catheters;
ii. balloons;
iii. other tubes;
iv. wires;
v. rods;
vi. threads; and
vii. other similar structures.
